(19) United States
(12) Patent Application Publication
(10) Pub. No.: US 2007/0032471 A1
(43) Pub. Date: Feb. 8, 2007

(54) Active substance combination comprising a 2,5-dihydroxybenzenesulfonic compound and a potassium ion channel modulator

(75) Inventors: Antoni Torrens Jover, Terrasa Barcelona (ES); Ines Alvarez Mathieu, Barcelona (ES); Inigo Saenz De Tejada Gorman, Madrid (ES); Javier Angulo Frutos, Valdemoro (ES); Helmut-Heinrich Buschmann, Esplugues de Llobregat Barcelona (ES)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: LABORATORIOS DEL DR. ESTEVE S.A., Barcelona (ES)

(21) Appl. No.: 10/566,132

(22) PCT Filed: Jul. 29, 2004
(86) PCT No.: PCT/EP04/08509

§ 371 Date: Jul. 7, 2006

(30) Foreign Application Priority Data
Jul. 30, 2003 (ES) P200301809

(51) Int. Cl. A61K 31/554; A61K 31/4184; A61K 31/185

(52) U.S. Cl. 514/211.07; 514/553; 514/355; 514/394

(57) ABSTRACT

The present invention relates to an active substance combination comprising at least one 2,5-dihydroxybenzenesulfonic compound and at least one K+ channel modulator, a medicament comprising said active substance combination, a pharmaceutical formulation comprising said active substance combination and the use of said active substance combination for the manufacture of a medicament.

The present invention relates to an active substance combination comprising at least one 2,5-dihydroxybenzenesulfonic compound and at least one potassium ion channel modulator, a medicament comprising said active substance combination, a pharmaceutical formulation comprising said active substance combination and the use of said active substance combination for the manufacture of a medicament.

Potassium ion (K⁺) channels play a crucial role in many physiological processes, e.g. in the regulation of vascular tone. Pharmacologically active substances that act as modulators for the K⁺ channel acitivity, such as K⁺ channel openers or K⁺ channel blockers, have consequently gained wide significance in the treatment of various K+channel related disorders, such as vascular diseases, diabetes or hypercholesterolemia.

Whereas conventional K⁺ channel modulators are effective in treating such K⁺ channel related disorders, in some instances they show undesirable side effects, which may range from unpleasant effects such as headache to life-threatening occurences such as cardiomyopathies.

It was therefore an object of the present invention to provide a medicament suitable for the prophylaxis and/or treatment of potassium ion (K⁺) channel related disorders, which preferably does not show the undesired side effects of known K⁺ modulators, or at least less frequent and/or less pronounced.

It has surprisingly been found that the pharmacological efficacy of K⁺ channel modulators may be enhanced by their administration in combination with one or more 2,5-dihydroxybenzenesulfonic compounds of general formula I given below. Consequently, the dose of the K⁺ channel modulator may be reduced and fewer, less pronounced to none undesired side effects occur.

Thus, one aspect of the present invention is an active substance combination comprising
(A) at least one 2,5-dihydroxybenzenesulfonic compound of general formula I, 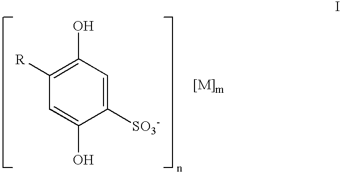
   wherein
R represents H or SO₃⁻,
M represents at least one cation,
n represents 1 or 2,
m represents 1 or 2,
optionally in form of a pharmaceutically acceptable solvate, and

(B) at least one K⁺ channel modulator.

The cation M in the 2,5-dihydroxybenzenesulfonic compounds of general formula I may be any physiologically acceptable cation known to those skilled in art, e.g. from P. Heinrich Stahl, Camille G. Wermuth (Editiors), “Handbook of Pharmaceutical Salts—Properties, Selections and Use”, Verlag Helvetica Chimica Acta, Zürich,

Switzerland, Wiley-VCH, Weinheim, Germany, 2002. The respective literature description is hereby incorporated by reference and is part of the disclosure. Those skilled in the art understand that the cation M has to be chosen in such a way that the overall charge of the 2,5-dihydroxybenzenesulfonic compounds of general formula I is neutral.

The present invention encompasses the use of a mixture of at least two of the afore mentioned 2,5-dihydroxybenzenesulfonic compounds of general formula I as well as mixed salts of these compounds, i.e. compounds with different cations M and/or different 2,5-dihydroxybenzenesulfonic residues as component (A).

Preferably the cation(s) M of the 2,5-dihydroxybenzenesulfonic compounds of general formula I is (are) selected from the group consisting of Ca²⁺, Mg²⁺, Na⁺, K⁺ and [NH₄₋ₓRₓ]⁺, wherein x is 0, 1, 2, 3 or 4 and R represents a branched or unbranched C₁₋₄-alkyl-radical. If x is greater than 1, i.e. if two or more alkyl-radicals are present in the [NH₄₋ₓRₓ]⁺-cation, they may be identical or different, whereby identical alkyl-radicals are preferred.

Preferably the active substance combination of the present invention may comprise one or more compounds selected from the group consisting of calcium 2,5-dihydroxybenzenesulfonate (calcium dobesilate), diethylamine 2,5-dihydroxybenzenesulfonate (ethamsylate) and bis(diethylamine)-2,5-dihydroxybenzene-1,4-disulfonate (persilate). Particularly preferably calcium 2,5-dihydroxybenzenesulfonate (calcium dobesilate) is used for the active substance combination according to the present invention.

The inventively used 2,5-dihydroxybenzenesulfonate compounds of general formula I may also be in the form of solvates, particularly in the form of hydrates. The manufacture of the 2,5-dihydroxybenzenesulfonate compounds of general formula I as well as their solvates may be accomplished by the use of reagents and methods known to those skilled in the art.

The manufacture of calcium 2,5-dihydroxybenzenesulfonate (calcium dobesilate) and diethylamine 2,5-dihydroxybenzenesulfonate (ethamsilate) is known, for example, from “The Merck Index”-13th edition, Merck & Co., R. Rahway, N.J., USA, 2001. Said literature description is hereby incorporated by reference and is part of the disclosure. The manufacture of bis(diethylamine) 2,5-dihydroxybenzene-1,4-disulfonate (persilate) is known, for example, from French Patent FR 73/17709 (Publication No. 2,201,888). The respective description is hereby incorporated by reference and is part of the disclosure.

According to the present invention any known K⁺ channel modulator may be used in the inventive active substance combination as component (B).

It is well known to those skilled in the art that different types and subtypes of K⁺ channels exist, e.g. from Christopher G. Sobey “Potassium Channel Function in Vascular Disease, Arterioscler. Throm. Vasc. Biol., January 2001, pages 28 ff, which is hereby incorporated by reference and forms part of the disclosure. Generally different K⁺ channel modulators show different activity for the different K⁺ channels. It can be tested by methods known to those skilled in the art, for which K⁺ channel a certain K⁺ channel modulator shows the best activity.

Preferably, the K⁺ channel modulator according to component B of the inventive active substance combination may be a K⁺ channel opener. K⁺ channel openers that may be used as component B as well as methods for their preparation are well known to those skilled in the art.

Preferably the inventive active substance combination comprises one or more K⁺channel openers selected from the group consisting of benzimidazole derivatives of general formula I, 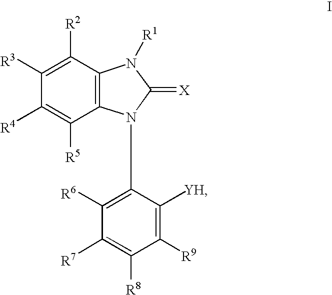
wherein

X represents O, S or NCN,

Y represents O or S,

R¹ represents hydrogen, NH₂ or branched or unbranched C₁₋₆alkyl,

R², R³, R⁴, R⁵ are each independently selected from the group consisting of hydrogen, halogen, CF₃, NO₂, NH₂, OH, C₁₋₆-alkoxy, C(═O)-phenyl or SO₂NR^{A}R^{B}, wherein R^{A} and R^{B}, identical or different, represent H or C₁₋₆-alkyl,

R⁶ represents hydrogen or NO₂,

R⁷ represents hydrogen, halogen, phenyl, CF₃ or NO₂, or

R⁸ represents hydrogen or NO₂,

or

R⁶ and R⁷ or R⁷ and R⁸ together with the two bridging carbon atoms from the phenyl ring form a C₄₋₇ carbocyclic ring, which may be saturated, unsaturated or aromatic,

R⁹ is hydrogen, halogen, NO₂ or SO₂NR^{A}R^{B}, wherein R^{A} and R^{B}, identical or different represent hydrogen or C₁₋₆-alkyl,

optionally in the form of a corresponding salt, or a corresponding solvate thereof, preferably the benzimidazole derivative of general formula I is 1-[2-Hydroxy-5-(trifluoromethyl)phenyl]-5-(trifluoromethyl)-1,3-dihydro-2H-benzimidazol-2-one (NS1619), 6-Amino-1,2-dihydro-1-hydroxy-2-imino-4-piperidinopyrimidine (minoxidil), (R)-(−)-2-[4-(4-Methyl-6-oxo-1,4,5,6,-tetrahydropyridazin-3-yl)phenylhydrazono]propanedinitrile (levosimendan), N-[2-Amino-4-(4-fluorobenzylamino)phenyl]carbamic acid ethyl ester (retigabine), (−)-3-[5-oxo-2-(trifluoromethyl)-1,4,5,6,7,8-hexahydroquinolin-4(S)-yl]benzonitrile (ZD-0947), 2-Amino-5-(2-fluorophenyl)-4-methyl-1 H-pyrrole-3-carbonitrile (NS-8), (3S, 4R)-3-Hydroxy-2,2-dimethyl-4-(2-oxopiperidin-1-yl)-N-phenyl-1-benzopyran-6-sulfonamide (KCO-912), (6-Chloro-3-(1-methylcyclopropylamino)-4H-thieno[3,2-e][1,2,4]thiadiazine-1,1-dioxide (NN-414), ABT-598, iptakalim hydrochloride, pinacidil, cromakalin, levcromakalin, aprikalim, N-(2-Hydroxyethyl)pyridine-3-carboxamide nitrate ester (nicorandil), (±)-(5-chloro-2-methoxyphenyl)-1,3-dihydro-3-fluoro-6-(trifluoromethyl)-2H-indol-2-one and ((3S)-(±)-(5-chloro-2-methoxyphenyl)-1,3-dihydro-3-fluoro-6-(trifluoromethyl)-2H-indol-2-one (also known as BMS-204352). More preferably NS1619 and/or pinacidil are used as K⁺ channel openers in the active substance combination according to the present invention.

The manufacture of the afore mentioned potassium ion channel openers is well known to those skilled in the art, e.g. for the benzimidazole derivatives from EP 0 477 818 A2, which is hereby incorporated by reference and forms part of the disclosure.

Preferably the inventive active substance combination comprises component (A) in an amount of 0.1 μM to 100 μM, more preferably 1 μM to 10 μM and the component (B) in an amount of 0.001 μM to 100 μM, more preferably 0.01 to 10 μM.

Also preferably, the inventive active substance combination comprises component (A) in an amount of 10 mg to 1000 mg, preferably 50 mg to 500 mg and component (B) in an amount of 1 mg to 100 mg, preferably 5 mg to 50 mg.

Another aspect of the present invention is a medicament comprising an inventive active substance combination and optionally at least one further active substance and/or optionally at least one auxiliary substance.

Said medicament is particularly suitable for the prophylaxis and/or treatment of male sexual dysfunction, preferably erectile dysfunction, female sexual dysfunction, hypertension, type I diabetes mellitus, type II diabetes mellitus, hypercholesterolemia, bladder instability, urinary incontinence, asthma, ischemic injury, ischemic insufficiency to the brain, cardiovascular diseases, preterm labor or for stopping labor preparatory to Caesarean delivery, stimulation of hair growth, epilepsy, gastrointestinal disorders including ulcers and dyspepsia, spasms, inflammations, inflammatory diseases and/or cancer.

The indication urinary incontinence includes also the indications imperative micturition (urge incontinence), hyperreflexia, urinary stress incontinence, mixed incontinence and Enuresis as well as others known to those skilled in the art.

For a more detailed description of these definitions and a standardisation of terminology reference is made to Abrams et al, Neurology and Urodynamics 21:167-178 (2002). The respective part of the description is hereby incorporated by reference and forms part of the disclosure.

Another aspect of the present invention Is the use of an inventive active substance combination for the manufacture of a medicament for the prophylaxis and/or treatment of male sexual dysfunction, preferably erectile dysfunction, female sexual dysfunction, hypertension, type I diabetes mellitus, type II diabetes mellitus, hypercholesterolemia, bladder instability, urinary incontinence, asthma, ischemic injury, ischemic insufficiency to the brain, cardiovascular diseases, preterm labor or for stopping labor preparatory to Caesarean delivery, stimulation of hair growth, epilepsy, gastrointestinal disorders including ulcers and dyspepsia, spasms, inflammations, inflammatory diseases and/or cancer.

Those skilled in the art understand that the components (A) and (B) of the active substance combination according to the present invention may be administered simultaneously or sequentially to one another, whereby in each case components (A) and (B) may be administerd via the same or different administration pathways, e.g. orally or parenterally. Preferably both components (A) and (B) are administered simultaneously in one and the same administration form.

Yet another aspect of the present invention are pharmaceutical formulations in different pharmaceutical forms comprising an inventive active substance combination and optionally at least one further active substance and/or optionally at least one auxiliary.

As well known to somebody skilled in the art the pharmaceutical formulations may—depending on their route of administration, also contain one or more auxiliary substances known to those skilled in the art.

The pharmaceutical formulations according to the present invention may be produced according to standard procedures known to those skilled in the art, e.g. from the tables of contents from “Pharmaceutics: the Science of Dosage Forms”, Second Edition, Aulton, M. E. (Ed.) Churchill Livingstone, Edinburgh (2002); “Encyclopedia of Pharmaceutical Technology”, Second Edition, Swarbrick, J. and Boylan J. C. (Eds.), Marcel Dekker, Inc. New York (2002); “Modem Pharmaceutics”, Fourth Edition, Banker G. S. and Rhodes C. T. (Eds.) Marcel Dekker, Inc. New York 2002 and “The Theory and Practice of Industrial Pharmacy”, Lachman L., Lieberman H. and Kanig J. (Eds.), Lea & Febiger, Philadelphia (1986). The respective descriptions are incorporated by reference and are part of the disclosure.

In a preferred embodiment of the present invention, the pharmaceutical formulation is suitable for oral administration.

If the pharmaceutical formulation is suitable for oral administration, it may preferably be in the form of a tablet, a capsule or a suspension.

The pharmaceutical formulation of the present invention for oral administration may also be in the form of multiparticulates, preferably pellets or granules, optionally compressed into a tablet, filled into a capsule or suspended in a suitable liquid. Suitable liquids are known to those skilled in the art.

In one embodiment of the present invention the pharmaceutical formulation comprises at least one of the components (A) and (B) at least partially in a sustained-release form.

By incorporating one or both of these components at least partially or completely in a sustained-release form it is possible to extend the duration of their effect, allowing for the beneficial effects of such a sustained release form, e.g. the maintenance of even concentrations in the blood.

Suitable sustained-release forms as well as materials and methods for their preparation are known to those skilled in the art, e.g. from the tables of contents from “Modified-Release Drug Delivery Technology”, Rathbone, M. J. Hadgraft, J. and Roberts, M. S. (Eds.), Marcel Dekker, Inc., New York (2002); “Handbook of Pharmaceutical Controlled Release Technology”, Wise, D. L. (Ed.), Marcel Dekker, Inc. New York, (2000); “Controlled Drug Delivery”, Vol. I, Basic Concepts, Bruck, S. D. (Ed.), CRC Press Inc., Boca Raton (1983) and from Takada, K. and Yoshikawa, H., “Oral Drug delivery”, Encyclopedia of Controlled Drug Delivery, Mathiowitz, E. (Ed.), John Wiley & Sons, Inc., New York (1999), Vol. 2, 728-742; Fix, J., “Oral drug delivery, small intestine and colon”, Encylopedia of Controlled Drug Delivery, Mathiowitz, E. (Ed.), John Wiley & Sons, Inc., New York (1999), Vol. 2, 698-728. The respective descriptions are incorporated by reference and are part of the disclosure.

If the pharmaceutical formulation according to the present invention comprises at least one of the components (A) and (B) at least partially in a sustained-release form, said sustained release may preferably be achieved by the application of at least one coating or provision of a matrix comprising at least one sustained-release material.

The sustained-release material Is preferably based on an optionally modified, water-insoluble, natural, semisynthetic or synthetic polymer, or a natural, semisynthetic or synthetic wax or fat or fatty alcohol or fatty acid, or on a mixture of at least two of these afore mentioned components.

The water-insoluble polymers used to produce a sustained-release material are preferably based on an acrylic resin, which is preferably selected from the group of poly(meth)acrylates, particularly preferably poly(C₁₋₄)alkyl(meth)acrylates, poly(C₁₋₄)dialkylamino(C₁₋₄)alkyl(meth)acrylates and/or copolymers or mixtures thereof, and very particularly preferably copolymers of ethyl acrylate and methyl methacrylate with a monomer molar ratio of 2:1 (Eudragit NE30D®), copolymers of ethyl acrylate, methyl methacrylate and trimethylammonium ethyl methacrylate-chloride with a monomer molar ratio of 1:2:0.1 (Eudragit RS®), copolymers of ethyl acrylate, methyl methacrylate and trimethylammonium ethyl methacrylate-chloride with a monomer molar ratio of 1:2:0.2 (Eudragit RL®), or a mixture of at least two of the above-mentioned copolymers. These coating materials are commercially available as 30 wt. % aqueous latex dispersions, i.e. as Eudragit RS30D®, Eudragit NE30D® or Eudragit RL30D®, and may also be used as such for coating purposes.

In another embodiment, the sustained-release material is based on water-insoluble cellulose derivatives, preferably alkyl celluloses, particularly preferably ethyl cellulose, or cellulose esters, e.g. cellulose acetate. Aqueous ethyl cellulose dispersions are commercially available, for example, under the trademarks Aquacoat® or Surelease®.

As natural, semisynthetic or synthetic waxes, fats or fatty alcohols, the sustained-release material may be based on carnauba wax, beeswax, glycerol monostearate, glycerol monobehenate, glycerol ditripalmitostearate, microcrystalline wax, cetyl alcohol, cetylstearyl alcohol or a mixture of at least two of these components.

The afore mentioned polymers of the sustained-release material may also comprise a conventional, physiologically acceptable plasticizer in amounts known to those skilled in the art.

Examples of suitable plasticizers are lipophilic diesters of a C₆-C₄₀ aliphatic or aromatic dicarboxylic acid and a C₁-C₈ aliphatic alcohol, e.g. dibutyl phthalate, diethyl phthalate, dibutyl sebacate or diethyl sebacate, hydrophilic or lipophilic citric acid esters, e.g. triethyl citrate, tributyl citrate, acetyltributyl citrate or acetyltriethyl citrate, polyethylene glycols, propylene glycol, glycerol esters, e.g. triacetin, Myvacet® (acetylated mono- and diglycerides, C₂₃H₄₄O₅ to C₂₅H₄₇O₇), medium-chain triglycerides (Miglyol®), oleic acid or mixtures of at least two of said plasticizers. Aqueous dispersions of Eudragit RS® and optionally Eudragit RL® preferably contain triethyl citrate. The sustained-release material may comprise one or more plasticisers in amounts of, for example, 5 to 50 wt. % based on the amount of polymer(s) used.

The sustained-release material may also contain other conventional auxiliary substances known to those skilled in the art, e.g. lubricants, coloured pigments or surfactants.

The pharmaceutical formulation of the present invention may also comprise at least one of the components (A) and (B) coverd by an enteric coating form which dissolves as a function of pH. Because of this coating, part or all of the pharmaceutical formulation can pass through the stomach undissolved and the components (A) and/or (B) are only released in the intestinal tract. The enteric coating preferably dissolves at a pH of between 5 and 7.5.

The enteric coating may be based on any enteric material known to those skilled in the art, e.g. on methacrylic acid/methyl methacrylate copolymers with a monomer molar ratio of 1:1 (Eudragit L®), methacrylic acid/methyl methacrylate copolymers with a monomer molar ratio of 1:2 (Eudragit S®), methacrylic acid/ethyl acrylate copolymers with a monomer molar ratio of 1:1 (Eudragit L30D-55®), methacrylic acid/methyl acrylate/methyl methacrylate copolymers with a monomer molar ratio of 7:3:1 (Eudragit FS®), shellac, hydroxypropyl methyl cellulose acetate-succinates, cellulose acetate-phthalates or a mixture of at least two of these components, which can optionally also be used in combination with the above-mentioned water-insoluble poly(meth)acrylates, preferably in combination with Eudragit NE30D® and/or Eudragit RL® and/or Eudragit RS®.

The coatings of the pharmaceutical formulations of the present invention may be applied by the conventional processes known to those skilled in the art, e.g. from Johnson, J. L., “Pharmaceutical tablet coating”, Coatings Technology Handbook (Second Edition), Satas, D. and Tracton, A. A. (Eds), Marcel Dekker, Inc. New York, (2001), 863-866; Carstensen, T.,“Coating Tablets in Advanced Pharmaceutical Solids”, Swarbrick, J. (Ed.), Marcel Dekker, Inc. New York (2001), 455-468; Leopold, C. S., “Coated dosage forms for colon-specific drug delivery Pharmaceutical Science & Technology Today, 2(5), 197-204 (1999), Rhodes, C. T. and Porter, S. C., Coatings, in Encyclopedia of Controlled Drug Delivery. Mathiowitz, E. (Ed.), John Wiley & Sons, Inc., New York (1999), Vol.1, 299-311. The respective descriptions are incorporated by reference and are part of the disclosure.

In another embodiment, the pharmaceutical formulation of the present invention contains one or both of components (A) and (B) not only in sustained-release form, but also in non-retarded form. By combination with the immediately released form, a high initial dose can be achieved for the rapid onset of the beneficial effect. The slow release from the sustained release form then prevents the beneficial effect from diminishing. Such a pharmaceutical formulation is particularly useful for the treatment of acute health problems.

This may be achieved, for example, by a pharmaceutical formulation having at least one immediate-release coating comprising at least one of the components (A) and (B) to provide for rapid onset of the beneficial effect after administration to the patient.

In another preferred embodiment of the present invention, the pharmaceutical formulation is suitable for parenteral administration, preferably intravenous administration.

Pharmacological Methods:

In-vitro-methods:

Vascular Reactivity of Human Penile Resistance Arteries

Penile small arteries, helicine arteries (lumen diameter 150-400 μm), which are the terminal branches of deep penile arteries, are dissected by carefully removing the adhering trabecular tissue, and arterial ring segments (2 mm long) and are subsequently mounted on two 40 μm wires on microvascular Halpern-Mulvany myographs (J. P. Trading, Aarhus, Denmark) for isometric tension recordings. The vessels are allowed to equilibrate for 30 min in physiological salt solution (PSS) of the following composition (mmol/l): NaCl 119, KCl 4.6, CaCl₂ 1.5, MgCl₂ 1.2, NaHCO₃ 24.9, glucose 11, KH₂PO₄ 1.2, EDTA 0.027 at 37° C. continuously bubbled with 95% 02/5% CO₂ mixture to maintain a pH of 7.4. Passive tension and internal circumference of vascular segments when relaxed in situ under a transmural pressure of 100 mmHg (L₁₀₀), are determined. The arteries are then set to an internal circumference equivalent to 90% of L₁₀₀, at which the force development was close to maximal (Mulvany & Halpern. Circ. Res. 41: 19-26, 1977). The preparations are then exposed to 125 mM K⁺ (KPSS, equimolar substitution of NaCl for KCl in PSS) and the contractile response is measured. The arteries are contracted with 1 μmol/l norepinephrine (80% of KPSS induced contraction approximately) and relaxation responses are evaluated by cumulative additions of compounds to the chambers. The arterial segments considered as lacking functional endothelium do not relax to 10 μmol/l acetylcholine.

Rat Mesenteric Resistance Arteries

Sprague-Dawley rats weighing 300-400 g are sacrificed by CO₂ inhalation. The mesentery is removed and placed in PSS. Third branch mesenteric arteries are dissected free of connective tissue under a light microscope and mounted as ring preparations on microvascular Halpern-Mulvany myographs. Isometric tension recording is performed as described for human penile resistance arteries.

Effects of Calcium Dobesilate on Relaxation Induced by K_{(ATP)}-Channel Activation in Human Penile Arteries:

Arterial segments are contracted with 1 μmol/I norepinephrine (NE) and, when a stable plateau is reached, arteries are exposed to the opener of ATP-sensitive K⁺-channels (K_{(ATP)}), pinacidil (1 nM to 1 mM). Reference is made to the corresponding part of the despription of Arena & Kass. Circ. Res., 65: 436-445, 1989, which forms part of the disclosure.

Then, arteries are washed and, after an equilibration period, treated or not (controls) with calcium dobesilate (10 μM) for 30 min. At this time, responses to pinacidil are again evaluated in NE-contracted arteries.

Effects of Calcium Dobesilate on Relaxation Induced by Ca²⁺-Activated K⁺-Channel Activation in Human Penile Arteries:

Arterial segments are contracted with 1 μmol/l norepinephrine (NE) and, when a stable plateau is reached, arteries are exposed to acetylcholine (ACh; 1 nM to 10 μM) to demonstrate the presence of endothelium. After a washout and equilibration period, preparations that relaxed in response to ACh are again contracted with NE and exposed to cumulative additions of the activator of Ca²⁺-activated K⁺-channel (K_{(ca)}), NS1619 (1 nM to 10 μM). Reference is made to the corresponding part of the description of Olesen et al. Eur. J. Pharmacol., 251: 53-59, 1994, which forms part of the disclosure. Then, arteries are washed and, after an equilibration period, treated or not (controls) with calcium dobesilate (10 μM) for 30 min. At this time, responses to NS1619 are again evaluated in NE-contracted arteries.

Effects of Calcium Dobesilate on Relaxation Induced by Ca²⁺-Activated K⁺-Channel Activation in Rat Mesenteric Arteries:

Arterial segments are contracted with 1 μmol/l norepinephrine (NE) and, when a stable plateau is reached, arteries are exposed to ACh (1 nM to 10 μM) to test the presence of endothelium. After a washout and equilibration period, preparations that relaxed in response to ACh are again contracted with NE and exposed to cumulative additions of the activator of Ca²⁺-activated K⁺-channel, NS1 619 (1 nM to 10 μM).

Then, arteries are washed and, after an equilibration period, treated or not (controls) with calcium dobesilate (10 μM) for 30 min. At this time, responses to NS1619 were again evaluated in NE-contracted arteries.

Those skilled in the art understand that the pharmacological methods described above for calcium dobesilate as component (A) and pinacidil or NS1619 as component (B) may analogously be carried out for other components (A) and/or (B).

In-vivo Methods:

The in-vivo activity of the active substance combination is tested as described in the reference of Saénz Tejada et al. in International Journal of Impotence Research 2003, 15, 90-93 under “Methods—Erectile responses to cavernosal nerve stimulation in anaesthetized rats”, which is hereby incorporated by reference and forms part of the disclosure.

The present invention is illustrated below with the aid of examples. These illustrations are given solely by way of example and do not limit the general spirit of the present invention.

### EXAMPLES

### Example 1

Hard Gelatin Capsule comprising calcium dobesilate and NS1619

| | | |
|---|---|---|
| Calcium dobesilate | 100 | mg |
| NS1619 | 30 | mg |
| Cellulose | 0.023 | g |
| Magnesiumstearate | 0.007 | g |
| Colloidal silicon dioxide | 0.005 | g |
| Total weight | 0.165 | g |

Calcium dobesilate, NS1619, Cellulose, Magnesiumstearate and Colloidal silicon dioxide in the afore mentioned amounts were thoroughly mixed in a conventional mixer and then filled into a conventional hard gelatin capsule.

### Example 2

Tablet comprising calcium dobesilate and Pinacidil

| | | |
|---|---|---|
| Calcium dobesilate | 100 | mg |
| Pinacidil | 30 | mg |
| Maize starch | 0.0650 | g |
| Lactose | 0.0520 | g |
| Povidone K-30 | 0.0175 | g |
| Citric acid monohydrate | 0.0125 | g |
| Magnesiumstearate | 0.0020 | g |
| Sodium bisulfite | 0.0010 | g |
| Total weight | 0.28 | g |

Calcium dobesilate, Pinacidil, Maize starch, Lactose, Povidone K-30, Citric acid monohydrate, Magnesiumstearate and Sodium bisulfite in the afore mentioned amounts were thoroughly mixed in a conventional mixer and then compressed into a tablet on a conventional tabletting press.

Pharmacological Methods and Data:

Human Penile Tissues:

Human penile tissue biopsies, were obtained from impotent men who gave informed consent at the time of penile prosthesis insertion. Tissues were maintained at 4-6° C. in M-400 solution (composition per 100 ml: manitol, 4.19 g; KH₂PO₄, 0.205 g; K₂HPO₄.3H₂O, 0.97 g; KCl, 0.112 g; NaHCO₃, 0.084 g) until used, which was between 2 and 16 hours from extraction. The corresponding part of Angulo et al., Br. J. Pharmacol, 136: 23-30, 2002 is hereby incorporated by reference and forms part of the disclosure.

Drugs and Materials:

Norepinephrine (arterenol), acetylcholine and NS1619 were obtained from Sigma Chemical Co. (St. Louis, Mo.). Pinacidil was obtained from RBI (Natwick, Mass.). Calcium dobesilate (DOBE) (calcium dihydroxy-2,5 benzenesulfonate, Doxium®) was provided by Dr. Esteve Laboratories (Barcelona, Spain). Drugs were dissolved in deionized water, except for NS1619 which was dissolved at 10 mmol/l concentration in DMSO. The subsequent dilutions were made in deionized water.

Data Analysis:

Relaxation responses are expressed as percentage of total relaxation (loss in tone) induced by the addition of 0.1 mmol/l papaverine HCl to the chambers at the end of the experiment. All data are expressed as mean±standard error. Complete concentration-response curves were obtained and compared by a two-factor analysis of variance (ANOVA) statistical test using StatView software for Apple computers.

The effect of calcium dobesilate on relaxation of human penile resistance arteries induced by pinacidil and in human penile resistance arteries and in rat mesenteric resistance artieries by NS1619 has been determined as described above.

It has been found that calcium dobesilate significantly potentiates relaxation of human penile resistance arteries induced by activation of K_{(ATP)} channels with pinacidil.

Furthermore, calcium dobesilate strongly potentiates relaxant responses induced by K_{(Ca)} channel activation in human penile resistance arteries.

This latter effect has also been found in rat mesenteric resistance arteries, where EDHF-(Endothelium-derived-hyperpolarization factor) mediated relaxation exists. Thus, calcium dobesilate enhances the efficiacy of K⁺ channel openers and thus of K⁺-channels, particularly of K_{(Ca)} channels.

The erectile response to cavemosal nerve electrical stimulation in anaesthetized diabetic rats was determined as described above.

It has been found that calcium dobesilate (10 mg/kg, intravenous administration) and the K⁺ channel opener NS1619 (0.3 mg/kg or 5 mg/kg, intravenous administration)—if administered alone—do not modify the erectile response in diabetic rats.

If an inventive active substance combination comprising calcium dobesilate (10 mg/kg) and the K⁺ channel opener NS1619 (0.3 mg/kg or 5 mg/kg) is intravenously administered to diabetic rats a significant improvement of the erectile response in diabetic rats is observed. Thus, a synergistic effect is found for the active substance combination according to the present invention.

1. Active substance combination comprising
(A) at least one 2,5-dihydroxybenzenesulfonic compound of general formula I, 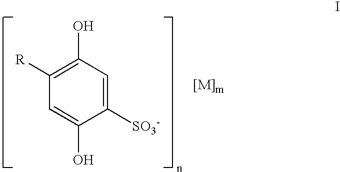
wherein
R represents H or SO₃⁻,
M represents at least one cation,
n represents 1 or 2,
m represents 1 or 2,
optionally in form of a pharmaceutically acceptable solvate, and
(B) at least one potassium ion (K⁺) channel modulator.

2. Active substance combination according to claim 1, characterised in that the cation(s) M is (are) selected from the group consisting of Ca²⁺, Mg²⁺, Na⁺, K⁺ and [NH_{4-X}R_{X}]⁺, whereby x is 0, 1, 2, 3 or 4 and R represents a branched or unbranched C₁₋₄-alkyl-radical that may be the same or different for x>1.

3. Active substance combination according to claim 1 or 2, characterized in that the compound of general formula I is calcium 2,5-dihydroxybenzenesulfonate (calcium dobesilate).

4. Active substance combination according to claim 1 or 2, characterized in that the compound of general formula I is diethylamine 2,5-dihydroxybenzenesulfonate (ethamsylate).

5. Active substance combination according to claim 1 or 2, characterized in that the compound of general formula I is bis(diethylamine)-2,5-dihydroxybenzene-1,4-disulfonate (persilate).

6. Active substance combination according to one or more of claims 1-5, characterized in that the modulator of component (B) is a K⁺ channel opener.

7. Active substance combination according to claim 6, characterized in that the K⁺ channel opener is selected from the group consisting of benzimidazole derivatives of general formula I, 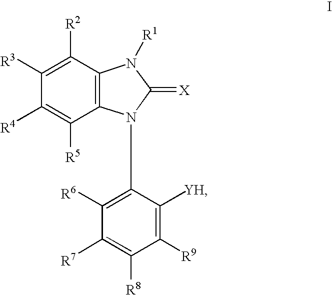
wherein
X represents O, S or NCN,
Y represents O or S,
R¹ represents hydrogen, NH₂ or branched or unbranched C₁₋₆-alkyl,
R², R³, R⁴, R⁵ are each independently selected from the group consisting of hydrogen, halogen, CF₃, NO₂, NH₂, OH, C₁₋₆-alkoxy, C(═O)-phenyl or SO₂NR^{A}R^{B}, wherein R^{A} and R^{B}, identical or different, represent H or C₁₋₆-alkyl,
R⁶ represents hydrogen or NO₂,
R⁷ represents hydrogen, halogen, phenyl, CF₃ or NO₂, or
R⁸ represents hydrogen or NO₂,
or
R⁶ and R⁷ or R⁷ and R⁸ together with the two bridging carbon atoms from the phenyl ring form a C₄₋₇ carbocyclic ring, which may be saturated, unsaturated or aromatic,
R⁹ is hydrogen, halogen, NO₂ or SO₂NR^{A}R^{B}, wherein R^{A} and R^{B}, identical or different represent hydrogen or C₁₋₆-alkyl,
optionally in the form of a corresponding salt, or a corresponding solvate thereof, preferably 1-[2-Hydroxy-5-(trifluoromethyl)phenyl]-5-(trifluoromethyl)-1,3-dihydro-2H-benzimidazol-2-one (NS1 619), 6-Amino-1,2-dihydro-1-hydroxy-2-imino-4-piperidinopyrimidine (minoxidil), (R)-(−)-2-[4-(4-Methyl-6-oxo-1,4,5,6,-tetrahydropyridazin-3-yl)phenylhydrazono]propanedinitrile (levosimendan), N-[2-Amino-4-(4-fluorobenzylamino)phenyl]carbamic acid ethyl ester (retigabine), (−)-3-[5-oxo-2-(trifluoromethyl)-1,4,5,6,7,8-hexahydroquinolin-4(S)-yl]benzonitrile (ZD-0947), 2-Amino-5-(2-fluorophenyl)-4-methyl-1H-pyrrole-3-carbonitrile (NS-8), (3S,4R)-3-Hydroxy-2,2-dimethyl-4-(2-oxopiperidin-1-yl)-N-phenyl-1-benzopyran-6-sulfonamide (KCO-912), (6-Chloro-3-(1-methylcyclopropylamino)-4H-thieno[3,2-e][1,2,4]thiadiazine-1,1-dioxide (NN-414), ABT-598, iptakalim hydrochloride, pinacidil, cromakalin, levcromakalin, aprikalim, N-(2-Hydroxyethyl)pyridine-3-carboxamide nitrate ester (nicorandil), (±)-(5-chloro-2-methoxyphenyl)-1,3-dihydro-3-fluoro-6-(trifluoromethyl)-2H-indol-2-one and ((3S)-(+)-(5-chloro-2-methoxyphenyl)-1,3-dihydro-3-fluoro-6-(trifluoromethyl)-2H-indol-2-one (BMS-204352), preferably from the group consisting of pinacidil and NS1619.

8. Active substance combination according to any one of claims 1 to 7, characterized in that it comprises component (A) in an amount of 0.1 μM to 100 μM, more preferably 1 μM to 10 μM.

9. Active substance combination according to claims 1 to 8, characterized in that it comprises component (B) in an amount of 0.001 μM to 100 μM, more preferably 0.01 to 10 μM.

10. Medicament comprising an active substance combination according to any one of claims 1 to 9 and optionally at least one further active substance and/or optionally at least one auxiliary.

11. Medicament according to claim 10 for the prophylaxis and/or treatment of male sexual dysfunction, preferably erectile dysfunction, female sexual dysfunction, hypertension, type I diabetes mellitus, type II diabetes mellitus, hypercholesterolemia, bladder instability, urinary incontinence, asthma, ischemic injury, ischemic insufficiency to the brain, cardiovascular diseases, preterm labor or for stopping labor preparatory to Caesarean delivery, alopecias, epilepsy, gastrointestinal disorders including ulcers and dyspepsia, spasms, preferably gastrointestinal spasms, inflammatory diseases, preferably gastrointestinal inflammation, and/or cancer.

12. Use of an active substance combination according to any one of claims 1 to 9 for the manufacture of a medicament for the prophylaxis and/or treatment of male sexual dysfunction, preferably erectile dysfunction.

13. Use of an active substance combination according to any one of claims 1 to 9 for the manufacture of a medicament for the prophylaxis and/or treatment of female sexual dysfunction.

14. Use of an active substance combination according to any one of claims 1 to 9 for the manufacture of a medicament for the prophylaxis and/or treatment of hypertension.

15. Use of an active substance combination according to any one of claims 1 to 9 for the manufacture of a medicament for the prophylaxis and/or treatment of type I diabetes mellitus and/or type II diabetes mellitus.

16. Use of an active substance combination according to any one of claims 1 to 9 for the manufacture of a medicament for the prophylaxis and/or treatment of hypercholesterolemia.

17. Use of an active substance combination according to any one of claims 1 to 9 for the manufacture of a medicament for the prophylaxis and/or treatment of bladder instability.

18. Use of an active substance combination according to any one of claims 1 to 9 for the manufacture of a medicament for the prophylaxis and/or treatment of urinary incontinence.

19. Use of an active substance combination according to any one of claims 1 to 9 for the manufacture of a medicament for the prophylaxis and/or treatment of asthma.

20. Use of an active substance combination according to any one of claims 1 to 9 for the manufacture of a medicament for the prophylaxis and/or treatment of ischemic injury.

21. Use of an active substance combination according to any one of claims 1 to 9 for the manufacture of a medicament for the prophylaxis and/or treatment of ischemic insufficiency to the brain.

22. Use of an active substance combination according to any one of claims 1 to 9 for the manufacture of a medicament for the prophylaxis and/or treatment of cardiovascular diseases.

23. Use of an active substance combination according to any one of claims 1 to 9 for the manufacture of a medicament for the prophylaxis and/or treatment of preterm labor.

24. Use of an active substance combination according to any one of claims 1 to 9 for the manufacture of a medicament for stopping labor preparatory to Caesarean delivery.

25. Use of an active substance combination according to any one of claims 1 to 9 for the manufacture of a medicament for the prophylaxis and/or treatment of alopecias.

26. Use of an active substance combination according to any one of claims 1 to 9 for the manufacture of a medicament for the prophylaxis and/or treatment of epilepsy.

27. Use of an active substance combination according to any one of claims 1 to 9 for the manufacture of a medicament for the prophylaxis and/or treatment of gastrointestinal disorders including ulcers and dyspepsia.

28. Use of an active substance combination according to any one of claims 1 to 9 for the manufacture of a medicament for the prophylaxis and/or treatment of gastrointestinal spasms.

29. Use of an active substance combination according to any one of claims 1 to 9 for the manufacture of a medicament for the prophylaxis and/or treatment of inflammatory diseases.

30. Use of an active substance combination according to any one of claims 1 to 9 for the manufacture of a medicament for the prophylaxis and/or treatment of gastrointestinal inflammation.

31. Use of an active substance combination according to any one of claims 1 to 9 for the manufacture of a medicament for the prophylaxis and/or treatment of cancer.

32. Pharmaceutical formulation comprising an active substance combination according to any one of claims 1 to 9 and optionally at least one further active substance and/or optionally at least one auxiliary.

33. Pharmaceutical formulation according to claim 32, characterized in that it is suitable for oral administration.

34. Pharmaceutical formulation according to claim 33, characterized in that the medicament is in the form of a tablet, a capsule or a suspension.

35. Pharmaceutical formulation according to claim 33, characterized in that is in form of multiparticulates, preferably pellets or granules, optionally compressed into a tablet, filled into a capsule or suspended in a suitable liquid.

36. Pharmaceutical formulation according to any one of claims 32 to 35, characterized in that it comprises component (A) and/or component (B) at least partially in a sustained-release form.

37. Pharmaceutical formulation according to claim 36, characterized in that it has at least one coating or matrix comprising at least one sustained-release material.

38. Pharmaceutical formulation according to claim 37, characterized in that the sustained-release material is based on an optionally modified, water-insoluble, natural, semisynthetic or synthetic polymer, or a natural, semisynthetic or synthetic wax or fat or fatty alcohol or fatty acid, or on a mixture of at least two of these afore mentioned components.

39. Pharmaceutical formulation according to claim 38, characterized in that the water-insoluble polymer is based on an acrylic resin, which is preferably selected from the group of poly(meth)acrylates, poly(C₁₋₄)dialkylamino(C₁₋₄)alkyl(meth)acrylates and/or copolymers thereof or a mixture of at least two of the afore-mentioned polymers.

40. Pharmaceutical formulation according to claim 38, characterized in that the water-insoluble polymers are cellulose derivatives, preferably alkyl cellulose and particularly preferably ethyl cellulose, or cellulose esters.

41. Pharmaceutical formulation according to claim 38, characterized in that the wax is carnauba wax, beeswax, glycerol monostearate, glycerol monobehenate, glycerol ditripalmitostearate, microcrystalline wax or a mixture of at least two of these components.

42. Pharmaceutical formulation according to any one of claims 38-41, characterized in that the polymers have been used in combination with one or more plasticizers.

43. Pharmaceutical formulation according to any one of claims 32 to 42, characterized in that it comprises at least one enteric coating.

44. Pharmaceutical formulation according to any one of claims 32 to 43, characterized in that it comprises at least one immediate-release coating comprising component (A) and/or component (B).

45. Pharmaceutical formulation according to claim 32 suitable for parenteral administration, preferably intravenous administration.
